(19) United States
(12) Patent Application Publication
(10) Pub. No.: US 2012/0171255 A1
(43) Pub. Date: Jul. 5, 2012

(54) SUBSTRATE IMPREGNATED WITH SPRAYED CLAY, AND METHOD FOR MANUFACTURING SAME

(76) Inventors: Valérie Fontas, Gressy-En-France (FR); Claude Martin, Sainte Maure (FR)

(21) Appl. No.: 13/382,356

(22) PCT Filed: Jul. 7, 2010
(86) PCT No.: PCT/FR2010/051441

§ 371 Date: Mar. 6, 2012

(30) Foreign Application Priority Data
Jul. 7, 2009 (FR) 0954696

(51) Int. Cl. B05D 3/02; D04H 13/00; A61K 33/06; B05D 1/02; A61K 9/00

(52) U.S. Cl. 424/400; 427/372.2; 427/210; 427/397.8; 424/684; 442/61; 428/219

(57) ABSTRACT

The invention relates to the production of a clay-impregnated substrate (10), including a step in which a pasty material (3), containing clay mixed with a liquid, is sprayed onto a substrate (1) having an aerated structure.

The invention relates to the production of a clay-impregnated substrate.

It therefore relates in particular in this case to the production of a part intended to cover an external area of a living organism, a human or an animal body, or a plant.

The use of clay for medical or cosmetic purposes has long been known, and the therapeutic or cosmetic properties of some clays have already been studied.

In this regard, in particular in EP-A-116 240, a strip with an active filler capable of being used in particular for human and animal treatment purposes has already been proposed. This strip includes a substrate with an aerated structure with a clay filler, in particular green clay, adhering to it.

U.S. Pat. No. 6,610,781, US 2007 0276308 and FR 2096036 refer to methods for impregnating clay in a substrate. U.S. Pat. No. 6,610,781 and US 2007 0276308 suggest a spray.

It is thus known to:
produce a pasty mixture containing a filler including (at least) essentially clay,
spray it toward a dry, flexible substrate with a fibrous structure,
then dry said substrate provided with this mixture.

None of these documents, however, provides truly convincing details for impregnating by spraying.

Moreover, both U.S. Pat. No. 6,610,781 and US 2007 0276308 cite various other modes of filling the substrate.

US 2007 0276308 indicates that, before being dried, the impregnated substrate must be rolled between compression rollers or scraped to further compress the mixture into the substrate.

And U.S. Pat. No. 6,610,781 requires the mixture that is to be placed in contact with the substrate to include a polymerization agent suitable for inducing polymerization in the mixture or in the presence of a substrate provided with the mixture.

This creates complex process conditions and proves that the impregnation solely by spraying is not mastered under conditions comparable to what is proposed here.

A problem solved here is therefore related to the conditions for performing, simply by spraying, the impregnation with a clay base filler (at least essentially) of a suitable substrate, so that the filler with which the dried substrate is provided does not become significantly separated from said substrate when it is then immersed in distilled water at ambient temperature.

To achieve this objective:
the pasty mixture is produced either with phyllitic, non-swelling clay, representing between 30% and 60% by weight of the mixture,
or the pasty mixture is produced in a tank, where an air pressure of between 1.5 and 7×10⁵ Pa has been established and/or the mixture is sprayed at a distance from the substrate of between 5 cm and 30 cm,
said substrate toward which the mixture is sprayed is stretched out, and is a nonwoven material with natural or synthetic fibers obtained by an aerodynamic and dry (airlaid) process, in which the mixture that is sprayed is free of polymerization agent capable of causing polymerization in the mixture or in the presence of the substrate provided with the sprayed mixture,
said mixture is sprayed so as to impregnate the substrate with it,
and, before being dried, the impregnated substrate is not rolled between compression rollers or scraped in order to further compress the mixture into the substrate.

Thus, a high-quality finished product will be obtained (preferably without edge irregularities), optionally with a multi-component filler, which adheres well, and without aggregation (good dispersion). The finished product can easily exist in various forms, without any notable changes. It is possible, if necessary, to vary the amount of filler, and to quickly go from a rolled strip production to a series of masks, patches, dressings, body towels and the like.

As other features, to be considered alone or in partial combinations, it is also recommended:
that the sprayed mixture have a clay mass fill ratio of less than 42%,
that it be sprayed against only one face of the substrate, capable of saturating it with filler, but without saturating said clay substrate throughout the entire thickness thereof,
that said impregnated substrate be dried on a bearing support that supports it on the side of its face opposite the side that has been sprayed with the mixture,
that a mixture free of diatomaceous earth be sprayed,
that the pasty mixture produced contain between 40% and 70% of liquid, in particular water, by weight,
that said nonwoven substrate (1) have:
dry and non-impregnated, a density of between 40 g/m² and 100 g/m² before spraying, and/or an absorption capacity (Edana test) of between 10 g/g and 18 g/g,
and between 80 g/m² and 550 g/m² impregnated, therefore after spraying;
that the pasty mixture be produced from clay having a particle size of between 10 microns and 100 microns;
that said substrate include only natural fibers including cellulose, the mixture to be sprayed including only natural compounds and thus free of any suspension additive, with agitation of the mixture to be sprayed being established in order to suspend the clay,
that the clay be an illite,
that said mixture be sprayed against only one face of the substrate, filling one face more than the opposite face, without saturating said substrate throughout the entire thickness thereof, and impregnating said filler in and between the fibers at least locally, in at least a portion of said thickness.

Concerning the product itself, in the same sense, it is possible to propose a flexible, rolled, dry product therefore including a porous airlaid substrate with natural or synthetic fibers and filled (at least) with clay, which filler is free of any chemically integrated network between the clay and a polymer and impregnates the substrate heterogeneously throughout the thickness thereof, in and between the fibers at least locally, with at least one non-saturation of one of the faces, and empty spaces existing between the fibers.

If the product is obtained by implementing the aforementioned method, the fibers of the filled substrate will be, at least locally, less entangled than those of the dry substrate, not yet filled, toward which the mixture will have been sprayed.

In general, the product in question may also be characterized advantageously in that:
the fibers of the filled substrate are less entangled on the side of the substrate opposite the side most filled,
said nonwoven substrate has, when impregnated, a density of between 80 g/m² and 550 g/m²,
the substrate is filled with a phyllitic, non-swelling clay with a particle size of between 20 microns and 30 microns, and preferably monophyllitic and polyphyllitic,
it is free of diatomaceous earth.

Other features and advantages will become clearer from the following non-limiting description with reference to the appended figures, in which:

FIG. 1 shows the possibility of sprinkling with a manual gun,

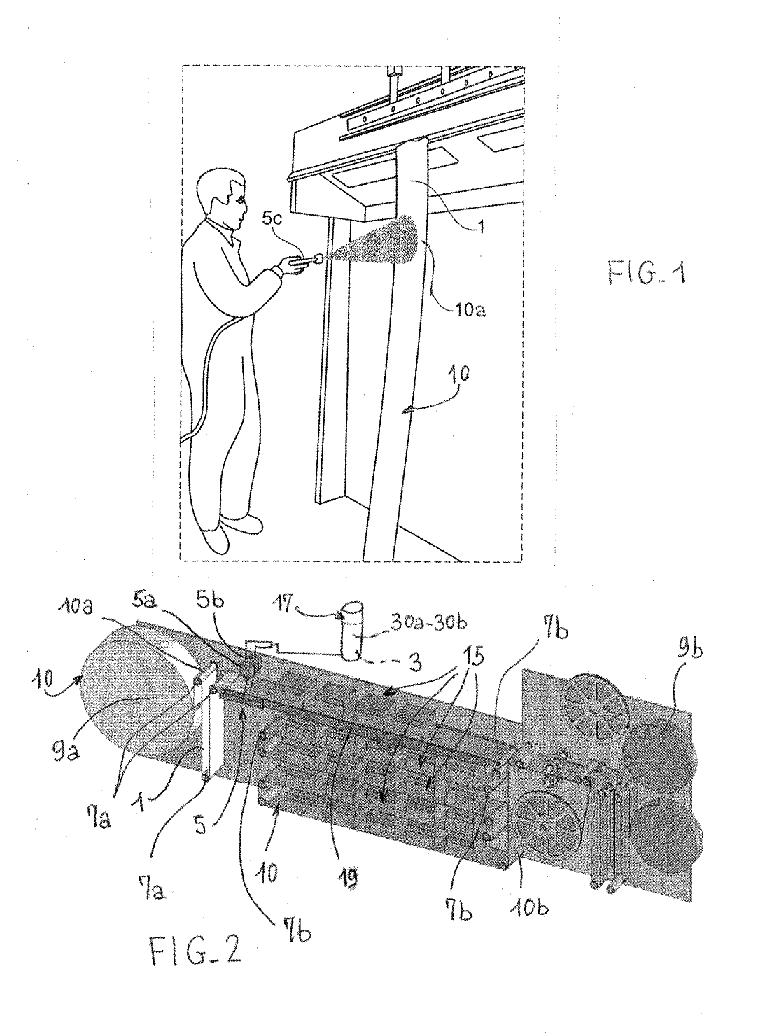

FIG. 2 diagrammatically shows an embodiment of an airlaid strip filled by automated spraying,

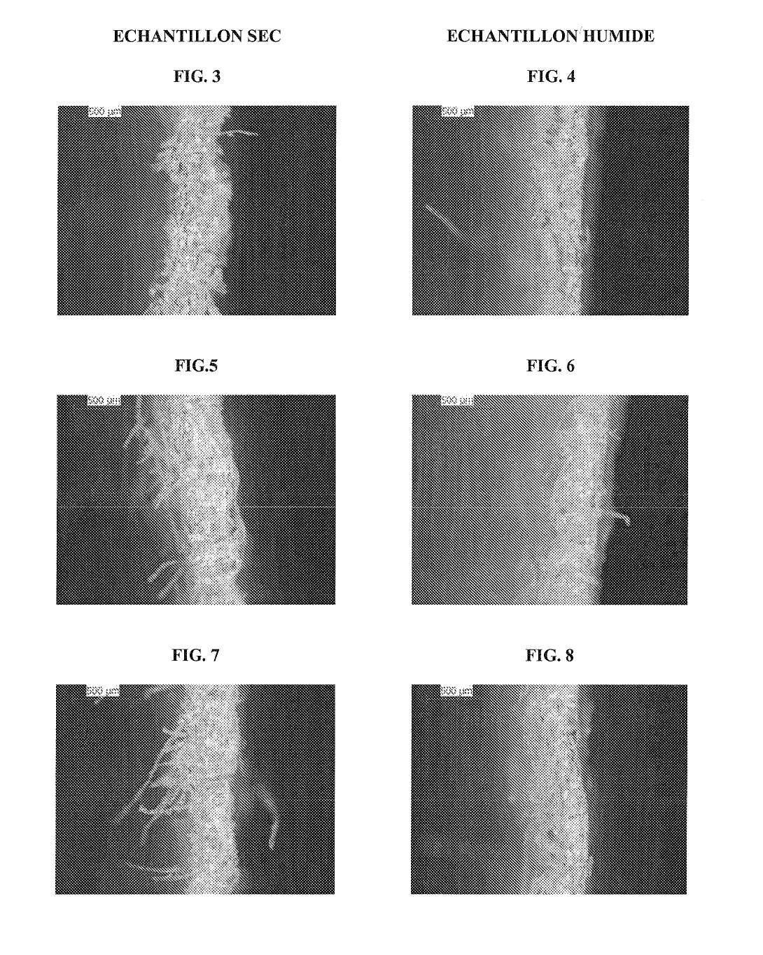

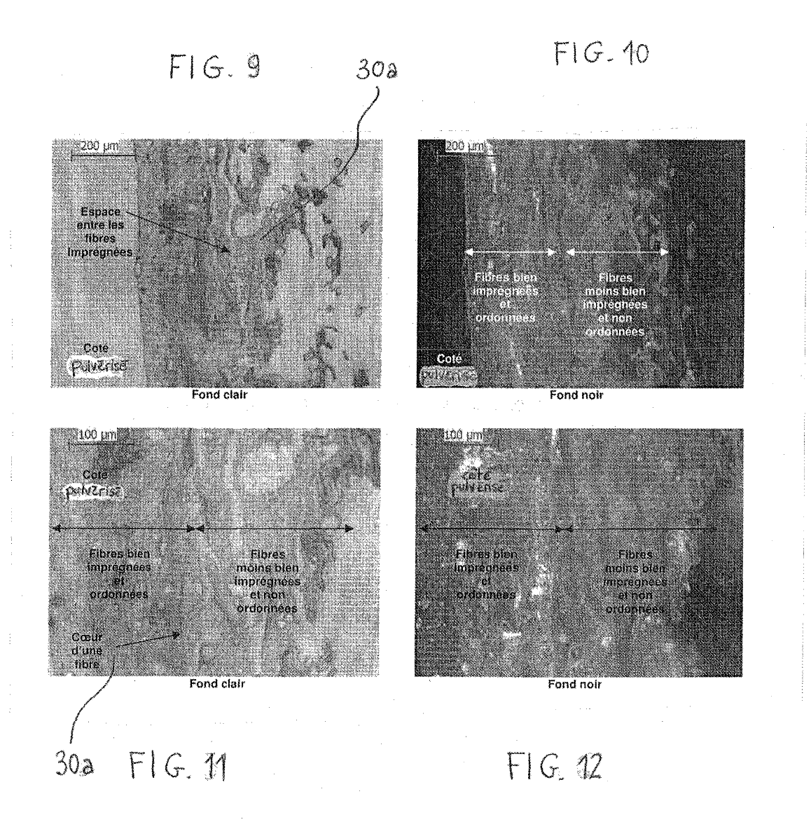

FIGS. 3, 4, 5, 6, 7 and 8 show, by groups of two (horizontally) cross-sections of a filled substrate (sprayed face at the right in each figure), with a polished cross-section, under optical microscope,

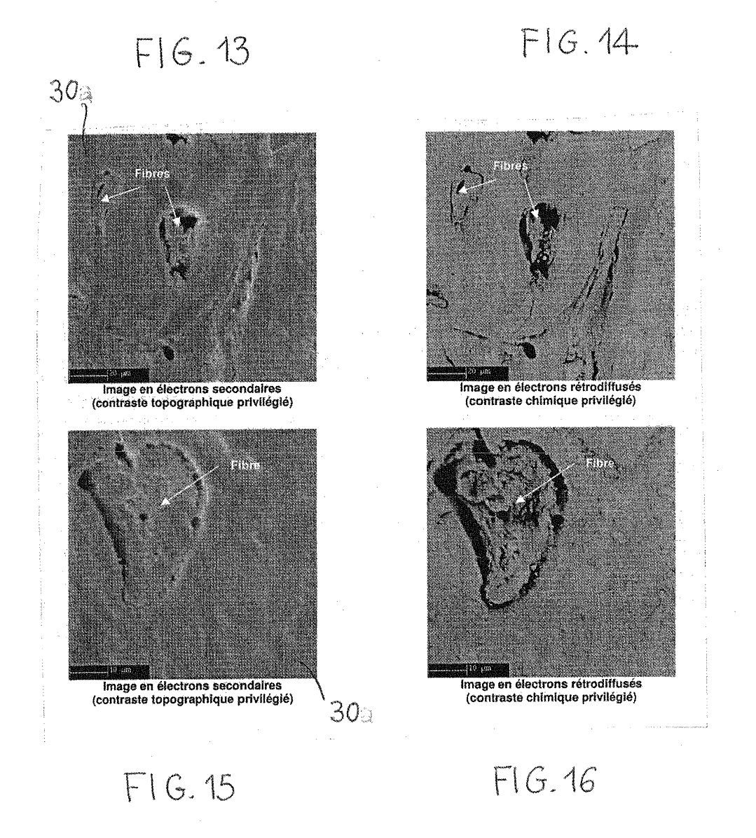

FIGS. 13, 14, 15 and 16 show a cross-section of a filled substrate, under a scanning microscope, with the fibers set in the clay, and

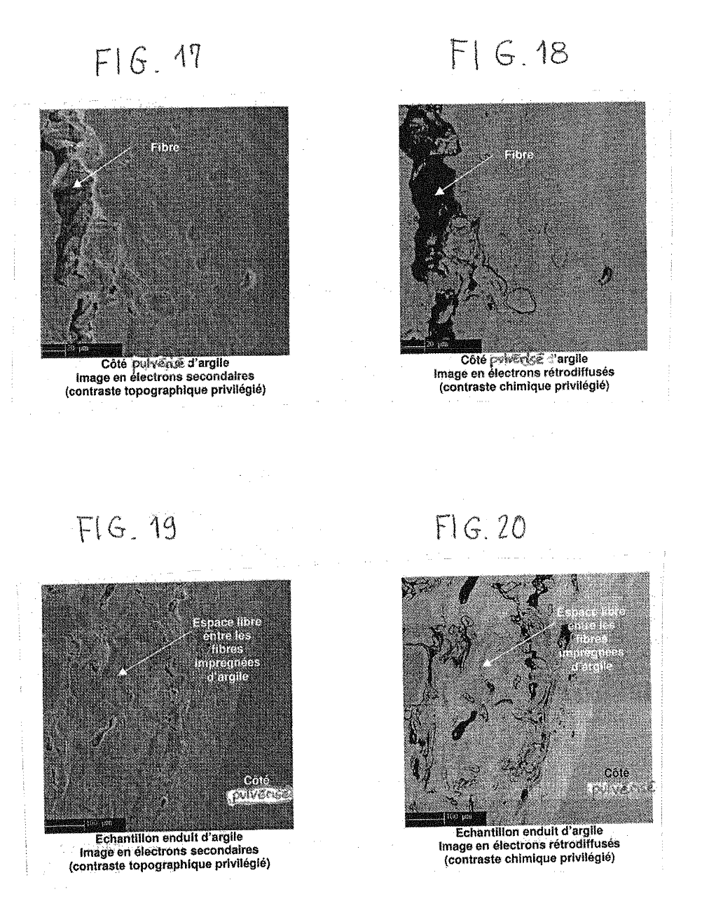

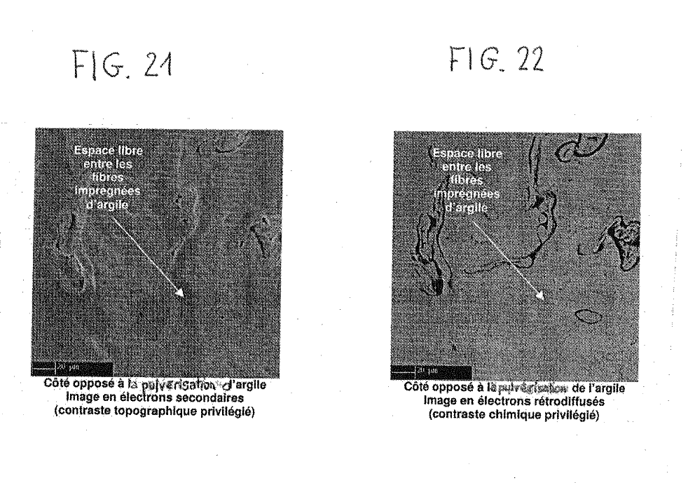

FIGS. 18, 19, 20, 21 and 22 show cross-sections of a filled substrate (sprayed face at the right in each figure), under scanning microscope.

What is presented below must enable the filler 30 not to separate significantly from the dried substrate 10 when it is then immersed in distilled water, at ambient temperature, as if it were being used by a customer.

To this end, and as shown in FIGS. 1 and 2, a flexible substrate 10 is proposed, which is impregnated (at least) with clay 30a (at the core and on the surface) by spraying a pasty product 3 containing the clay in a mixture with a liquid 30b, on a flexible clean substrate 1 with an aerated structure.

To produce this pasty product, the clay is supplied dry, and in the form of particles.

To impregnate the core of the substrate 1, the pasty product to be sprayed is a paste in this case containing, by weight of the mixture, between 30 and 60% clay and between 40 and 70% liquid, in particular water, or even alcohol, or a combination thereof. The clay 30a used will be phyllitic and non-swelling.

A particularly effective result has been observed with a clay having a particle size of between 10 microns and 100 microns, and preferably between 20 microns and 30 microns. It is recommended that the clay to be sprayed be illite.

Additives for reinforcing the properties of the clay may be added to said pasty mixture, such as plant extracts, aromas, etc. The clay may remain largely predominant, and even constitute 90%.

To promote the spraying, it is recommended that the pasty product 3 contain a mass fill ratio of clay of less than 42%. In addition, in the tank 17, it will have been held in suspension favorably by agitation (preferably with dispersants, such as “darvan”™); a rotating-arm agitator, which stirs the muddy paste at low speed.

It should be noted that the mixture to be sprayed, and therefore the filled substrate obtained, may conveniently be free of diatomaceous earth, contrary to the recommendations of US 2007 0276308.

In addition, the mixture will be free of any polymerization agent capable of causing polymerization in the mixture or in the presence of the substrate provided with the sprayed mixture. Due to the features disclosed here, it is indeed unnecessary to produce a clay-polymer network composite as in U.S. Pat. No. 6,610,781 in order for the filler to hold to the substrate under the expected conditions.

To promote the quality of the products in the context of use in the food, hygiene or care product industry, as well as the recycling of the products after use, it is recommended:
that the substrate 1 toward which the spray is directed include only natural fibers including (preferably exclusively) cellulose,
that the mixture to be sprayed include only natural compounds and that it thus be free of any suspension additive, as the aforementioned agitator enables sufficient agitation to be established in order to place the clay, and more generally the filler to be sprayed, in suspension.

For the spraying, it is also recommended:
that the pasty mixture be produced in a tank 17 where an air pressure of between 1.5 and 7×10⁵ Pa has been established,
and/or that the mixture be sprayed at a distance from the substrate of between 5 cm and 50 cm (preferably 30 cm for strips, FIG. 2).

The combination of the two features ensures the quality of the result.

Concerning the feeding means, FIG. 2 shows that the spraying is performed over a horizontal substrate strip 1, with vertical nozzles, such as 5a and 5b (FIG. 2), spraying the mixture downward, and belonging to pneumatic means 5 connected to the interior of the tank 17.

An air inlet pressure in the/each nozzle on the order of 1.8 to 2.5×10⁵ Pa is favorable. “Devilbiss”™ nozzles operating under 3×10⁵ Pa of air pressure with a muddy mixture and 1.4-mm nozzles are suitable.

The manual version, shown in FIG. 1, with an operator maneuvering a spray gun 5c directed toward the substrate 1, which is then vertical, is not recommended.

The spraying is directed toward a substrate kept substantially stretched out.

FIG. 1, tension means 7a, 7b and reels such as 9a and 9b ensure the feeding of the substrate, taken up and kept stretched out.

Favorably, the spraying is performed by additionally filling, at 30, a single one of the faces (in this case identical) 10a of the substrate, and without saturating said substrate throughout the entire thickness thereof.

For this, the spraying will take place only on one side of the substrate.

As shown in the tests presented below, it is thus recommended to spray the mixture only toward one face of the substrate 10, until it is filled, in principle, to at least 60 g/m² of clay, without of course saturating the opposite face 10b, or said substrate throughout the entire thickness thereof, by impregnating instead the filler 30 in and between the fibers at least locally, in at least a portion of said thickness.

To obtain an effective product (good impregnation and hold of the filler, surface homogeneity, quality of cut-outs of the filled product if there are any), a clean absorbent substrate 1 is used, which is stretched out and dry, and is a nonwoven material made of natural or synthetic fibers, obtained by an aerodynamic and airlaid process.

As described in detail, for example, on the website: “http://cerig.efpg.inpg.fr/tutoriel/non-tisse/page03.htm”, the airlaid process is a dry process consisting of transporting and dispersing fibers of the type mentioned above in an air flow. Typically, the fibers are supplied and passed through perforated rotary cylinders or distribution systems in order to form a porous, aerated web on a conveyor belt (distribution chamber located above a belt with a vacuum system incorporated below the belt).

To achieve, under the best conditions, the desired impregnation, and in particular the results indicated below, it is recommended that the substrate 1 have:
when dry and non-impregnated, a density of between 40 g/m² and 100 g/m² before spraying, and/or an absorption capacity (Edana test) of between 10 g/g and 18 g/g,
and between 80 g/m² and 550 g/m² impregnated, therefore after spraying.

FIG. 2 shows that, after the spraying, the substrate impregnated by the filler will very advantageously be passed through, or between, drying means 15, with the specification that, before this, unlike what is taught in US 2007 0276308, the impregnated substrate will not have been rolled (pressed) between compression rollers or scraped in order to further compress the mixture into the substrate.

For the desired impregnation quality, including in the test of the filled substrate, dried, then immersed in distilled water, at ambient temperature, it is recommended that the impregnated product be dried on a bearing support 19 (substantially horizontal) that supports it on the side of its face 10b opposite the side toward which the mixture is sprayed.

The losses of a portion of the filler during drying, and therefore of the finished product, will be limited on the unsprayed side 10b (in this case, therefore, the lower side).

The drying may include drying means, in particular a chamber, where the filled substrate, in this case the flattened strip, will be dried at around 80 to 200° C. for 5 to 15 minutes.

Thus, the filled strip rolled in 9b will be dry.

To check the quality of the results, tests were conducted, as described in detail below, with examinations of cross-sections of materials, on segments:

### Type of Analyses:

clean airlaid substrate densities between 50 g/m² and 85 g/m², with thicknesses of between 0.45 mm and 1.4 mm, without preparation, then after coating in a transparent resin and polishing,
optical microscope with enlargement of 25 to 500,
materials (substrates) analyzed: clean airlaids, then impregnated,
guns with nozzles of Ø 1.8 mm,
pressures in the mud tank (17): between 1.5 and 7×10⁵ Pa,
sprayed fillers: phyllitic, non-swelling clay (illite) representing between 30% and 41% by weight of the mixture. The liquid was water.
Base weights measured:
clean airlaid: see above,
impregnated airlaid: 490 g/m²;
impregnation by the sprayed filler, under the aforementioned conditions, on a single face of the substrate 1.
Observation: these measurements were performed on samples of 1 dm2 and do not take into account variations produced by spraying with a manual gun.

### Results:

### Observations Without Preparation

Non-impregnated airlaid substrate: Entangled fibers; Average diameter of a fiber: 20 to 30 μm (typically 25 μm)
Impregnated substrate: Thickness: lower (on the order of −15 to more than −20% (20.6%) with respect to the dry, clean airlaid substrate); Clay in the thickness of the airlaid; Neater fibers, less entangled than in the dry, clean substrate, at least on the side opposite the side having been sprayed.

It is noted that the dry sample (impregnated, then dried naturally by resting on a support on the unsprayed side) has lost a small but insignificant amount of the clay filler (see FIGS. 3, 5, 7, where the fibers are clearly impregnated), which “losses” are then even more limited in the wetting of the first sample in the water of a beaker (for 1 to 2 min); see FIGS. 4, 6 and 8.

Observations with Preparations (Inclusion in a Polished Resin Block)
Non-impregnated airlaid substrate: messy arrangement of fibers, same average fiber diameter: 25 μm
Impregnated substrate: Fibers neater over 300 to 450 μm (sprayed side), a little less on the opposite face;

Total impregnation of the substrate, but no saturation aside from the sprayed face and over around 80 to 120 μm.

### CONCLUSIONS

The clay filler is distributed in the thickness of the airlaid (with gradients), although it is sprayed on only one side. The face opposite the spray contains little clay after drying.
Reduction in thickness: The spray pressure causes a decrease in the thickness of the airlaid material. The shrinkage of the clay during drying can also be an explanation: the distance between the sheets of the silicated microstructure is reduced.
Neatness and cohesion of fibers: The fibers appear to be rearranged by the pressure, the action of the spraying liquid and the final drying. The assembly becomes cohesive.

The drawings of FIGS. 3-22 thus confirm the above, in the context of a nonwoven airlaid substrate with cellulosic fibers.

It is seen that the clay-based filler impregnates the substrate heterogeneously throughout the entire thickness thereof, in and between the fibers at least locally, with saturation on the side of one of the two faces of the substrate and in a first portion of the thickness, and non-saturation of the opposite face, where there are empty spaces between the fibers.

Observations without Preparation (as above), under said Optical Microscope (FIGS. 3-8)

The decrease in the thickness appears to be due to better cohesion of the fibers with one another owing to the clay impregnation and the pressure induced by the spraying action, which enables the fibers to be rearranged more neatly.

After drying, the fibers on the side opposite the sprayed side are (almost) no longer coated with clay particles.

It is also observed that, on the side opposite the saturated side, the fibers appear to be neater.

Observations with Preparation/Inclusion in a Polished Resin Block (as above), with said Optical Microscope; see FIGS. 9 to 12:

Coated with clay, the airlaid fibers appear to be in order over around 370 μm of the impregnated side: these are the fibers best coated with clay. In this portion, the contact between the clay and the fibers is close: no porosity has been observed. On the unsprayed side, separations between clay-impregnated fibers are distinguished: they appear to be disordered.

### PARTIAL CONCLUSIONS

When the clay is sprayed on the cellulosic fiber tissue, it is observed that:
the fibers are covered with clay throughout the entire thickness of the tissue;
the fibers opposite the sprayed side are less covered with clay particles;
the clay gives cohesion to the assembly owing to close contact between the fibers and the clay. The cohesion between the fibers and the clay as well as the fact that these clay particles are sprayed enable the cellulosic fibers to become ordered. In this way, the thickness of the tissue decreases.

When the clay-coated tissue is wetted, the fibers least impregnated with clay lose some of their clay particles. However, the fibers keep a certain cohesion and the thickness of the tissue remains generally identical.

Observations under the Electronic Microscope (see FIGS. 13 to 22):

Procedure:

1) Carbon metallization of a polished cross-section as above;

2) Observations under scanning electron microscopy.

The observation of a cross-section of an impregnated sample, with a thickness of 500 μm, enables three zones to be distinguished:
the first zone, on the side where the clay is sprayed on the tissue, consists of a very compact layer of around 100 μm;
the second zone consists of a less compact mass of clay-covered fibers, over around 300 μm. In this mass, there are spaces between the fibers;
the third zone, opposite the impregnation side, is the one containing sparse clay-impregnated fibers (around 200 μm).

Each fiber observed in a cross-section is completely impregnated with clay. However, the thickness of the clay layer varies: it is greater on the side where the clay has been sprayed. The clay appears to have penetrated to the core of the fibers, resulting in close contact between the two products.

### CONCLUSIONS

This microscopy makes it possible to specify that the clay is first deposited in the airlaid substrate, has saturated it and then is stacked on the direct spraying face. This deposition is not homogeneous throughout the thickness: first, there is a fiber-free compact layer of around 100 μm, then, over 300 μm, is a fiber/clay mixture in which the fibers are totally embedded in the clay (little material is missing). Finally, a zone corresponding to the face opposite the spraying has more material missing and is not saturated with filler.

The clay embeds all of the fibers and seals the surface exposed to the spraying, thus causing a quasi-pure filler layer to form.

The penetration of the filler into the fibers causes a close attachment between the fibers and the filler.

Thus, the clay filler remains trapped in the dried airlaid (penetration into and between the fibers) and is deposited in a fine layer on the substrate during the wetting of the sample (clay between the fibers driven by the spray liquid).

Under conditions similar to those described above, it has also been verified that comparable results can still be obtained by spraying only on a face of the airlaid substrate, and filling this sprayed face with 60 g/m², then 180 g/m² of clay, therefore without saturating it.

A lower filled substrate thickness of −5% to more than −20% with respect to the dry, clean airlaid substrate is noted overall.

In every case, the filler does not significantly separate from the dried filled substrate when it is then immersed in distilled water, for between 3 s and 120 s, at ambient temperature (between 20° C. and 30° C., typically 25° C.). These tests conducted, samples impregnated then dried under lamps, resting on the unsprayed side, then with a time of complete immersion in distilled water for 3 s, 5 s, 7 s, 15 s and 30 s, successively, did not show notable (significant) separation between the airlaid substrate and the filler, therefore the clay, in particular by deposition in water.

To best take advantage of the qualities of the clay in particular in terms of preservation of agri-food products or action by skin or even subcutaneous contact (wounds, hemostatic performance, etc.), care/beauty/hygiene (including sexual hygiene), it is clearly recommended, after the tests performed, that the clay impregnating the nonwoven airlaid substrate 1 be an illite (phyllitic structure), comprised of 60 to 75% illite, 20 to 30% smectite, 5 to 10% kaolinite and various trace mineral elements (in particular magnesium, potassium, zinc, copper and manganese).

This will in principle be completed with:
a substrate 1 with natural fibers (cellulose) only,
and a clay consisting integrally of a natural mixture (unwashed or heat-treated or ionized in particular) having a particle size of between 20 microns and 30 microns, and of which the average diameter of the pores will be 70 to 75 Angstroms.

Thus, with this illite (in this case, natural hydrous aluminum silicate, commonly called green clay) having a porosity of 0.11 to 0.13 cc/g, the specific surface will enable optimal adsorption.

For the uses specified, it will also be noted that the following clays are suitable: green, pink, yellow and red clays.

The field of products for phytosanitary use (plants, gardening, etc.) and applications in the preservation of agri-food products are also covered and beneficial.

Concerning cut-outs of the dried, finished product, they are possible without heating the cutting means, and without fraying. The product can even be torn easily, while remaining strong enough for the envisaged uses, in particular in the wet state.

The visible layer of deposited clay does not become worn during the reeling and cutting operations or other (dry) transformation operations, even if the filled substrate, which remains flexible, is folded.

1. A method for producing a clay-impregnated substrate, including steps in which:
a pasty mixture is produced, containing a filler including essentially clay,
the pasty mixture is sprayed toward a dry, flexible substrate having a fibrous structure,
then said substrate provided with said mixture is dried, wherein, with a view to preventing the filler from becoming significantly separated from the dried substrate when it is then immersed in distilled water, at ambient temperature:
the pasty mixture produced is made of phyllitic, non-swelling clay, representing between 30% and 60% by weight of the mixture,
at least one of the following conditions is implemented:
the pasty mixture is produced in a tank where an air pressure of between 1.5 and 7×10⁵ Pa is established, and,
the mixture is sprayed at a distance from the substrate of between 5 cm and 30 cm,
said substrate toward which the mixture is sprayed is stretched out, and is a nonwoven material having at least one of natural and synthetic fibers obtained by an aerodynamic and dry process, in which the mixture that is sprayed is free of polymerization agent capable of causing polymerization in one of the mixture and the substrate provided with the sprayed mixture,
said mixture is sprayed so as to impregnate the substrate with it,
and, before being dried, the impregnated substrate is neither rolled between compression rollers nor scraped in order to further incorporate the mixture into the substrate.

2. Method according to claim 1, wherein the mixture that is sprayed has a clay mass fill ratio of less than 42%.

3. The method according to claim 1, wherein the spraying is performed by filling one face more than an opposite face of the substrate, without saturating said substrate throughout the entire thickness thereof.

4. The method according to claim 3, wherein said impregnated substrate is dried on a bearing support that supports it on a first face (10b) opposite a second face toward which the mixture is sprayed.

5. The method according to claim 1, wherein the sprayed pasty mixture is free from diatomaceous earth.

6. The method according to claim 1, wherein the pasty mixture (3) that is produced contains between 40% and 70% liquid by weight.

7. The method according to claim 1, wherein said nonwoven substrate (1) has:
when dry and non-impregnated, at least one of:
a density of between 40 g/m² and 100 g/m² before spraying, and,
an absorption capacity of between 10 g/g and 18 g/g,
and a density of between 80 g/m² and 550 g/m² impregnated, after spraying.

8. The method according to claim 1, wherein the pasty mixture is produced with clay having a particle size of between 20 microns and 30 microns.

9. The method according to claim 1, wherein:
said nonwoven substrate toward which the spraying is directed includes only natural fibers including cellulose,
the mixture which is sprayed out includes only natural compounds and is thus free of any suspension additive, and said mixture is agitated to be sprayed, in order to suspend the clay.

10. The method according to claim 1, wherein said clay is an illite.

11. The method according to claim 1, wherein said mixture is sprayed against one face of the substrate;
without saturating at least one of an opposite face and at least a portion of the thickness of the substrate, and,
while impregnating the filler in and between the fibers at least locally, over at least a portion of said thickness.

12. A flexible, dry, rollable product, including a nonwoven porous substrate having at least one of natural and synthetic fibers, filled at least essentially with clay and having a thickness and two opposite faces, in which the filler impregnates the substrate heterogeneously throughout said thickness, in and between the fibers at least locally, with a non-saturation of at least one of said the faces with empty spaces existing between the fibers, wherein the substrate is obtained by an aerodynamic and dry process, said filler being free of any chemically integrated network between clay and a polymer.

13. The product according to claim 12, wherein one of the faces is less filled at least essentially with clay than the opposite face, and the fibers of the filled substrate are less entangled on said less-filled face.

14. The product according to claim 12, wherein said nonwoven substrate has, when impregnated, a density of between 80 g/m² and 550 g/m² impregnated.

15. The product according to claim 12, wherein the substrate is filled by a non-swelling phyllitic clay.

16. The product according to one of claims 12 to 15, characterized in that it is free of diatomaceous earth.

17. (canceled)

18. The product according to claim 12, wherein the clay is an illite including 60 to 75% illite, 20 to 30% smectite and 5 to 10% kaolinite.

19. The product according to claim 13, wherein the clay is an illite consisting integrally of a natural mixture having a particle size of between 20 microns and 30 microns, and of which the average diameter of the pores is 70 to 75 Angstroms.

20. The product according to claim 13, wherein the fibers of the filled substrate are less entangled on the side of the substrate opposite the side most filled.

21. A flexible, dry, rollable product, including a nonwoven porous substrate having at least one of natural and synthetic fibers, filled at least essentially with clay and having a thickness and two opposite faces, in which the filler impregnates the substrate heterogeneously through said thickness, in and between the fibers at least locally, with a non-saturation of at least one of said the faces, with empty spaces existing between the fibers, wherein the substrate is obtained by an aerodynamic and dry process, and one of said faces is less filled at least essentially with clay than the opposite face and the fibers of the filled substrate are less entangled on said less-filled face.
